(19) United States
(12) Patent Application Publication
(10) Pub. No.: US 2012/0203127 A1
(43) Pub. Date: Aug. 9, 2012

(54) NASAL CANNULA WITH INTEGRATED THERMAL FLOW SENSING

(75) Inventor: Noam Hadas, Tel Aviv (IL)

(73) Assignee: SLP LTD., Tel Aviv (IL)

(21) Appl. No.: 13/021,791

(22) Filed: Feb. 7, 2011

(51) Int. Cl. A61B 5/087

(52) U.S. Cl. 600/537

(57) ABSTRACT

A device and method for providing an airflow sensor apparatus including a nasal cannula nosepiece body, an oral prong extending therefrom and a temperature sensor, integrated therewith. The temperature sensor includes an electrically-conductive elastomer based sensor material such as electrically-conductive ink, electrically-conductive Form-In-Place elastomer material and electrically-conductive elastomer wire.

### FIELD AND BACKGROUND OF THE INVENTION

The present invention relates to a nasal cannula and, more particularly, to a nasal cannula with an integrated thermal flow sensor.

Sleep apnea is a sleep disorder characterized by having multiple pauses in breathing or shallow breaths during sleep. Each pause in breathing, called an apnea, or significant deduction in respiration flow, called a hypopnea, can last from 10 seconds to minutes, and may occur 50 times or more an hour.

A typical sleep test includes an overnight stay in a sleep center where the patient is fitted with numerous different sensors to monitor various functions during sleep. The sleep study, or Polysomnography (PSG), is a comprehensive recording of the biophysiological changes that occur during sleep. The PSG monitors many body functions including brain activity (EEG), eye movements (EOG), muscle activity or skeletal muscle activation (EMG) and heart rhythm (ECG) during sleep. After the identification of the sleep disorder sleep apnea in the 1970s, the breathing functions respiratory airflow and respiratory effort indicators were added along with peripheral pulse oximetry. Respiratory airflow is the major indicator used to determine sleep apnea.

Nasal and oral airflow is normally measured using a pressure transducer connected to a nasal cannula, and/or by a thermal sensor placed in or near the nostrils; the pressure transducer is considered the more sensitive for detecting decreases in respiratory airflow termed hypopneas, while the thermal sensor is considered better for detecting complete cessations of flow, or apneas. This allows the clinician/researcher to measure the amplitude of respiration and identify interruptions in breathing. The American Academy of Sleep Medicine has determined that both airflow pressure and temperature should ideally be recorded in order to present sufficient parameters for analysis of the disorder: “The sensor to detect apnea is an oronasal thermal sensor and to detect hypopnea is a nasal pressure transducer. Ideally, [portable monitors] should use both sensor types” (“Clinical Guidelines for the Use of Unattended Potable Monitors in the Diagnosis of Obstructive Sleep Apnea in Adult Patients” published in the Journal of Clinical Sleep Medicine vol. 3 No. 7, 2007, pg. 741). This practice has been widely adopted. Applying both sensors under the nose is problematic even to a trained sleep technician. With the shift toward at-home testing, there is a need to make the diagnostic equipment more ambulatory and easier for the layman to apply.

Various attempts have been made to improve flow sensors for sleep disorders. U.S. Pat. No. 5,573,004, to Groenke describes a thermal flow sensor, but relates to double-sided printing of the resistive element to increase stability to flexion of the substrate. This patent describes a standalone thermal sensor that is not, and cannot be [comfortably] integrated with a nasal cannula for sampling respiratory pressure. Furthermore, the silk-screening fabrication is laborious, necessitating at least the alignment of the conductive ink and silver ink, which are applied in separate steps. This manufacture method is costly due to both the manufacturing steps as well as the silver ink material which is used merely as a connector.

U.S. Pat. No. 5,190,048 to Wilkinson describes a similar device that of Groenke, only the fabrication method is different, using discrete thermal sensors on a flexible substrate connected with a copper line. The Wilkinson patent suffers from at least the same drawbacks as Groenke.

U.S. Pat. No. 6,165,133 to Rapoport et al. describes thermal flow sensors applied externally on a pressure sampling nasal cannula. This patent describes the coupling of two standalone devices for a single use. The method of coupling standalone components is commonly used in the field of sleep disorder assessment. The application of the sensor bundle is complex and intricate (and not very comfortable for the user) and needs to be handled by a professional trained in the field. Furthermore, it is unclear how the invention maintains galvanic insulation between the thermocouple and user.

It would be highly advantageous to have a nasal cannula for sensing pressure which has a thermal sensor (thermistor or thermocouple) integrated therein, allowing the user (as opposed to a technician) to apply both sensors in one step, insuring proper placement and alignment, and preventing the need to disassemble the assembly and prepare the thermal sensor for reuse (a cannula is usually used only once, whereas the thermal sensor is generally more expensive to produce and therefore reused after sterilization). It would further be advantageous to have an integrated thermal sensor which is manufactured in a single step, using cost effective materials without undue use of non-sensor material, and does not suffer from the abovementioned drawbacks of the prior art.

### SUMMARY OF THE INVENTION

According to the present invention there is provided an airflow sensor apparatus including: (a) a nasal cannula nosepiece body; (b) an oral prong extending from the nosepiece body; and (c) a temperature sensor, integrated with the nasal cannula nosepiece body and the oral prong.

Heretofore, no known prior art has succeeded in integrating a temperature sensor with a nasal cannula. All prior attempts have been to couple the two incompatible technologies together in a bundle. This ‘bundle’ is complex and intricate, difficult to apply to a user and uncomfortable to wear on the face. The currently proposed solution makes use of the conductive properties of the thermal sensor materials in a heretofore unexplored manner (namely the placement of the thermistor) in the field of sleep disorders.

According to further features in preferred embodiments of the invention described below the temperature sensor includes an electrically-conductive elastomer based sensor material selected from the group of: (i) an electrically-conductive ink; (ii) an electrically-conductive Form-In-Place elastomer; and (iii) an electrically-conductive elastomer wire.

According to still further features in the described preferred embodiments the electrically-conductive ink sensor is printed on the nosepiece body and the oral prong.

According to still further features in the described preferred embodiments the electrically-conductive ink material is mechanically compatible with the nosepiece body. Seeing as the PVC material of the cannula is soft, flexible and stretchable, the printed ink or Form-In-Place material must also be flexible and stretchable so that when the cannula is stretched or flexed (during application or while be worn), the conductive material does not become disconnected or dislodges from the cannula.

According to still further features the apparatus further includes a pair of electrically-conductive ink contact areas, conductively coupled to the printed thermal sensor, for conductively coupling mechanically attached external sensor leads to the printed thermal sensor.

According to still further features the apparatus further includes a pair of circular abutments operationally coupled to a proximal surface of the nasal cannula nosepiece body, for intervening between the contact areas and a face of a user.

According to still further features the apparatus further includes an insulation substrate, insulating the electrically-conductive ink thermal sensor; and a second electrically-conductive ink thermal sensor, printed on the insulation substrate, wherein the electrically-conductive ink thermal sensors and the insulation substrate combine to form a temperature-responsive capacitor.

According to further features in preferred embodiments of the invention described below the temperature sensor includes a sealed volume tube threaded through the nosepiece body, and oral prong, wherein the sealed volume tube is operable to be connected to a pressure transducer, and wherein temperature changes effect air pressure changes within the sealed volume tube, the air pressure changes can to be measured by the pressure transducer.

According to the present invention there is provided a method for preparing a nasal cannula with integrated thermal sensor, including the steps of: providing the nasal cannula; and (b) applying electrically-conductive, temperature sensitive, elastomer material to the nasal cannula.

According to further features in preferred embodiments the electrically-conductive, temperature sensitive, elastomer material is selected from the group including: (i) an electrically-conductive ink; (ii) an electrically-conductive Form-In-Place elastomer; and (iii) an electrically-conductive elastomer wire.

According to further features in preferred embodiments the application is effected by a process selected from the group including: (a) a pad printing process; (b) a silk-screening process; (c) direct placement of Form-In-Place elastomer material by robotic 3D dispenser; and (d) manual threading of the conductive elastomer wire through the nosepiece.

### BRIEF DESCRIPTION OF THE DRAWINGS

Various embodiments are herein described, by way of example only, with reference to the accompanying drawings, wherein:

FIG. 1 is a front view of a preferred embodiment of the current invention;

FIG. 2 is a side view of a preferred embodiment of the current invention;

FIG. 3 is an isometric front view of a preferred embodiment of the intention;

FIG. 4 is a front view of a second preferred embodiment of the invention;

FIG. 5 is a front view of a third preferred embodiment of the invention;

FIG. 6 is a front view of a forth preferred embodiment of the invention.

### DESCRIPTION OF THE PREFERRED EMBODIMENTS

The principles and operation of an airflow sensing apparatus according to the present invention may be better understood with reference to the drawings and the accompanying description.

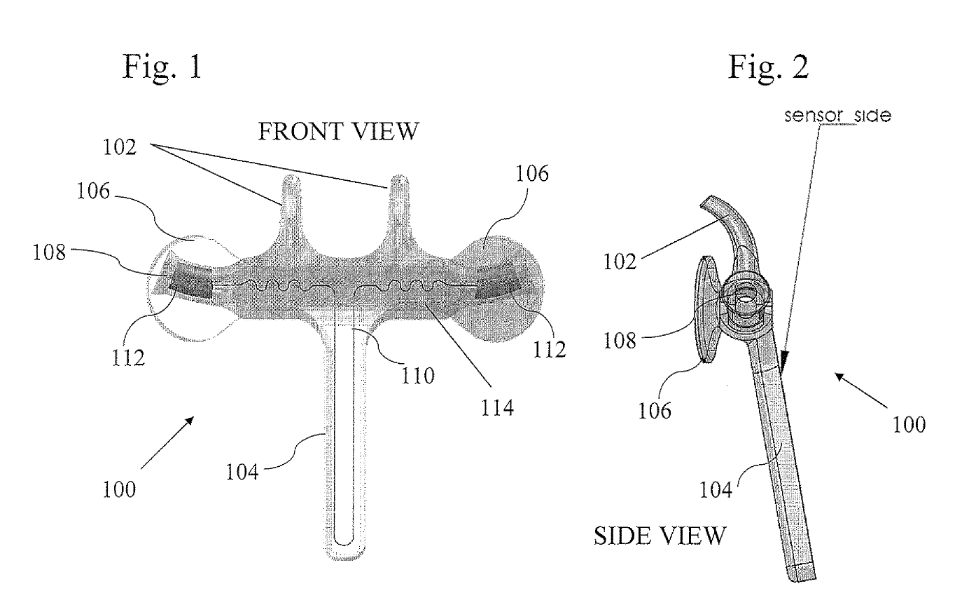

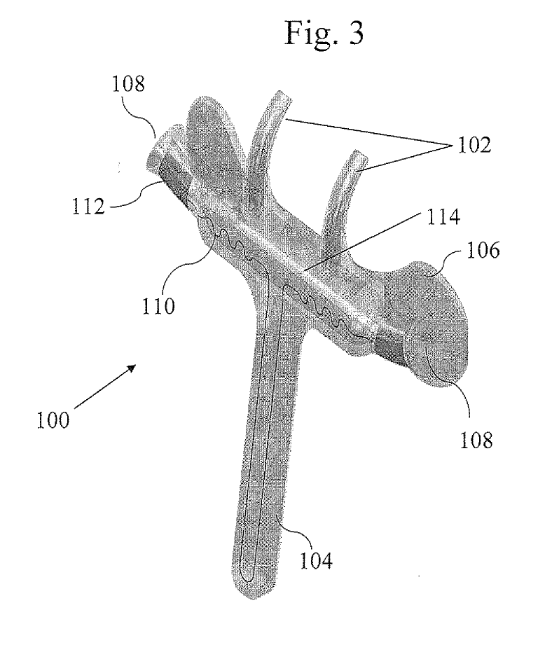

Referring now to the drawings, FIGS. 1-3 illustrate a preferred embodiment of the current invention. FIG. 1 is a front view of an embodiment of the invention and. FIG. 2 is a side view of embodiment of the invention of FIG. 1. FIG. 3 is an isometric front view of the invention of FIG. 1. Apparatus 100 is an inventive nosepiece of a nasal cannula with nasal prongs 102 and oral prong 104. The ‘nosepiece body’ is the horizontal section 114 of the cannula from which the nasal and oral prongs extend. The term ‘nosepiece’, ‘nasal cannula’ or ‘nasal cannula nosepiece’ are all used interchangeably to refer to the nosepiece body and prongs, as well as, in some embodiments, the circular abutments. The innovative nosepiece of the current invention replaces the standard nose piece of a standard cannula and connects to two side tubes (not shown) for transmitting the sampled pressure changes to the pressure transducer. The innovatively integrated thermal sensors of the current invention are made from electrically-conductive elastomer material. In some embodiments of the invention the electrically-conductive elastomeric material is an electrically-conductive ink and in some other embodiments the electrically-conductive elastomer material is an electrically-conductive elastomeric wire.

Oral prong 104, is operable to extend over the upper lip of the user, exposing a thermal sensor—which is innovatively integrated therein—to changes in ambient temperature resulting from oral respiration of the user. Note that unlike standard oral sampling cannulas, in the current embodiment the oral prong is not hollow, and is not used to sample air pressure in front of the mouth.

In the embodiment of the invention depicted in FIGS. 1-3, the innovative integrated sensor is a printed electrically-conductive ink thermistor 110 also interchangeably referred to herein as a line sensor. Conductive ink changes resistance with changes in temperature. Exhalation heats the ink and inhalation cools the ink. The changing temperature of the conductive ink can be recorded by noting the respective changes in electrical resistance of the conductive ink line. One exemplary type of conductive ink suitable to be used as a thermistor is carbon ink DS 119-28 manufactured by Creative Materials, Inc., Mass., USA. Carbon ink 119-28 is an extremely flexible, pad printable, electrically, conductive, carbon filled ink, suitable for application by pad printing, dipping and syringe dispensing. This product features excellent adhesion to Kapton, Mylar, glass and a variety of other surfaces. Unlike many other conductive materials, this product is very resistant to flexing and creasing. Any such conductive inks or conductive elastomers with similar attributes as those enumerated above are included within the scope of the invention. The conductive ink does not come into contact with the skin of a user and therefore does not require either insulation or skin contact biocompatibility. Of course, insulation can be added where desired.

The conductive ink thermistor 110 is printed along the length of the nosepiece body (that is intended to lie beneath the nostrils of the user) and along the oral prong 104. The printed thermistor substantially covers the horizontal/latitudinal length of cannula exposed to temperature changes resulting from nasal respiration. The distributed sensor is an improvement of over prior art sensors printed on the nasal prongs. In the prior art, such as U.S. Pat. No. 5,251,636 to Neuman, or U.S. Pat. No. 5,161,541 to Bowman et al., thermistors are printed on the nasal prongs. If the prongs become misaligned or moved out of the nostrils (e.g. when the user turns to the side during sleep), the fidelity of the signal can be significantly compromised. This is not the case in the current invention, as the distributed line sensor/conductive ink thermistor provides a larger area for sensing nasal flow than the nasal prongs alone.

The section of the electrically-conductive ink thermistor printed on the oral prong senses temperature changes resulting from oral respiration. Thermistor contact areas 112 are printed on the nasal cannula on each end of the thermistor. In use, monitoring leads (not shown) are attached to contact areas 112 with connector clips (not shown) or some other conductive means. For instance, the connector clip can be a U shaped contact made of phosphor-bronze or copper, and soldered to a lead wire. The wires connect to the interface circuit (not shown) which translates changes in resistance due to changes in respiratory air flowing over the sensor line. The wires are routed along the cannula tubes over both ears and under the chin. A pair of circular abutments 106 (described further below) serve to insulate the connector clips (or other connective means) from skin contact, resolving biocompatibility concerns. A very low current is passed through the thermistor and the changes in the conductivity of the ink are recorded. The thermistor helps record the regularity and intensity of the user's breathing.

In an alternative embodiment (not shown), the same conductive ink sensor line is printed so that the line both begins and ends on the same side of the cannula body. In this embodiment, one contact pad may be on the front of the nose piece and the second one is on the back of the nose piece (insulated from the user by the circular abutments). The two wires needed to connect to the sensor line are routed from the same side of the cannula, potentially minimizing wire entanglement and simplifying the arrangement of the system on the patient. Alternatively, the conductive ink line may be printed along the tubes on both sides of the cannula, and the electrical connection to the interface circuit made far from the face, under the chin or even near the pressure transducer connection.

In some embodiments of the invention, the edges of the cannula are tapered, increasing in diameter towards the nosepiece ends (near the connection points 108), so that the connector clips are prevented from sliding off contact areas 112 during use. Other similar structural innovations made to the cannula for the same or similar purpose as mentioned above, are included in the scope of the invention. Standard cannula tubing are intended to remain permanently attached to connection points 108. In the current embodiment of the invention, the nosepiece, including the oral prong, has a substantially flat outer surface. A flat or flattened surface is more convenient to print on with conductive ink than the standard, tubular nosepiece. On the other hand, a standard nasal cannula can also be printed on, due in part to the versatility of the pad-printing process and in part to the flexibility of the PVC medium. The result of the pad-printing process, whether on a flat or tubular surface, is an equally effective product. The pad-printing process can be performed in a single step (including printing contact areas 112), directly onto the cannula. Previously known methods such as ‘silk screening’, are less viable methods for printing on not-flat surfaces such as a cannula. In some methods known in the art, such as those employed in U.S. Pat. No. 5,161,541 to Bowman et al., two separate materials are used in the production process: carbon ink and silver ink. This process requires at least two separate steps in the production process, one step for each material. Furthermore, the two materials have to be carefully aligned. The pad-printing of electrically-conductive carbon-ink directly onto the cannula significantly simplifies the process by using only one material (carbon ink) as opposed to the two or more materials (each necessitating a separate step in the application process). Furthermore, silver inks are many times more expensive than carbon ink. In the current invention, no silver ink is needed. The innovative process for printing directly on a cannula is a lower cost, simplified and quicker process.

In some of the preferred embodiments, substantially circular abutments 106 provide additional comfort to the user. Furthermore, the abutments (as well as the line printing) assist the user in identifying the correct side of the cannula to place against the face (this is not always easily discernable with standard nasal cannulae). If the cannula is placed the wrong way, the curved nasal prongs are miss-aligned with the nasal flow and the resulting signal is problematic. A pressure transducer (not shown) senses airflow pressure in the cannula.

Potentially, a company logo can be printed on the cannula in conductive ink, so long as the logo forms an uninterrupted line from one contact to the other. The logo serves the double purpose as a thermistor on the one hand, and an advertisement on the other. When higher sensitivity is desired, the sensor line can be formed wave-like, as depicted in FIGS. 1-3.

Due to the flexible and stretchable nature of the cannula, the cannula is often stretched when being arranged on a patient or during use. It is therefore preferable to print on the PVC medium of the cannula with a type of carbon ink which is both flexible and stretchable. In the event that the cannula is stretched or twisted, the flexible ink will remain adhered to the cannula and will not chip off or disrupt the signal.

In the current embodiment of the invention, depicted in FIGS. 1-3, oral prong 104 is tilted distally from the vertical by 5-10 degrees, in order to minimize friction of prong with the upper lip of a user. Furthermore the oral prong is aligned with the external edge of the nosepiece tube in order to further remove the prong from the skin and upper lip. In FIGS. 1-3 the oral prong is not hollow and does not sense pressure. It is hereby made clear that a hollow oral prong, operable to sample oral respiration pressure, is included in the scope of the invention. The oral prong can be further made bendable with an internal or external stiffener, so that the user can reposition the prong to lie in the optimal position in front of the mouth. The tubular form of a hollow oral prong does not prevent or hamper the pad-printing process, as explained above.

In an alternative embodiment, the integrated sensor includes a ‘Form-In-Place’ (FIP) conductive gasket material, which is generally applied to the nosepiece in an automated process. An automated FIP gasketing process applies programmed shapes of conductive elastomer gasket material on to metal or plastic substrates using a dispenser attached to a robotic arm.

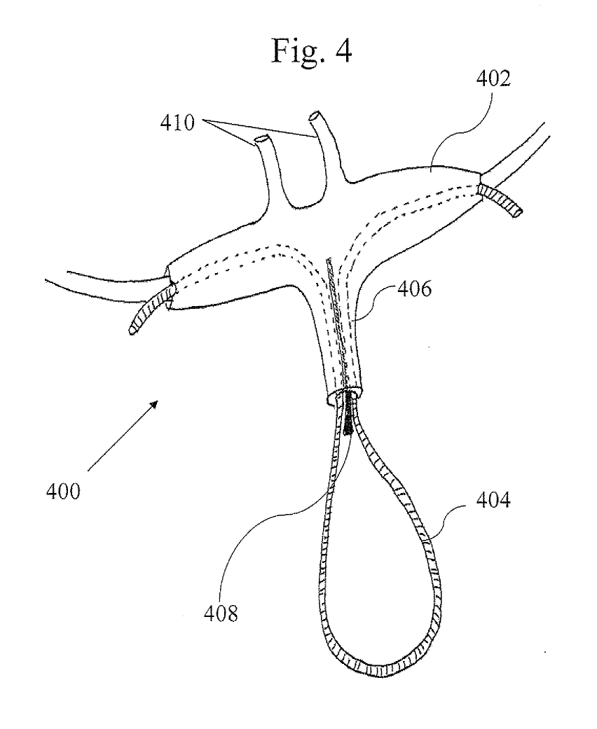

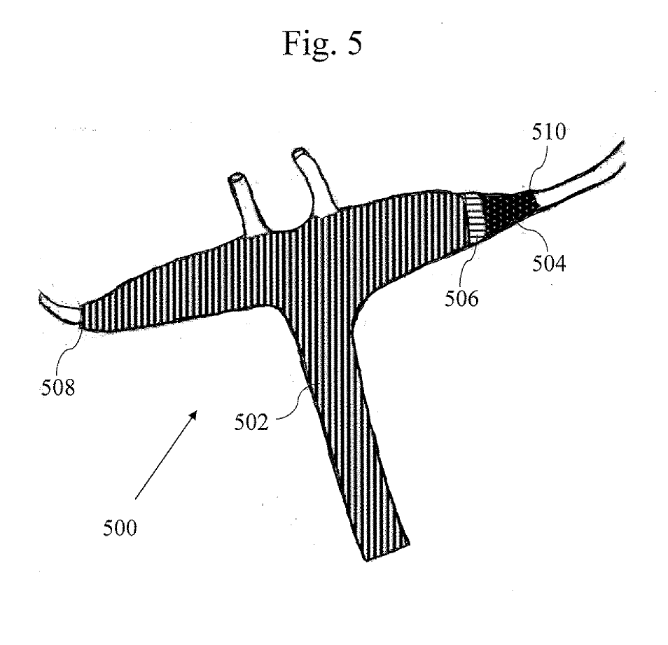

Another possible configuration is shown in FIG. 4. FIG. 4 is a front view of a preferred embodiment of an innovative nasal cannula nosepiece with an integrated thermal airflow sensor 400. In the current embodiment, the thermal sensor 404 is a carbon-loaded, extruded, silicone wire which, like conductive ink, is an electrically-conductive elastomer material that changes resistance with temperature changes. The silicone sensor wire is loaded with carbon nano-particles which have the property of temperature-related resistance. This is a novel use of sensor material not previously used in the field of sleep disorder testing. Sensor wire 404 is threaded through the nasal cannula nosepiece 402 so that a loop of the wire protrudes out of the oral prong 406. An insulator strip 408 prevents the two segments of wire from touching each other. The sensor wire 404 senses temperature changes in two areas: nasal respiration effects temperature changes to the sensor wire inside the cannula, via nasal prongs 410; oral respiration effects changes to the loop segment of the wire, which protrudes out of the oral prong. One possible method for preparing the threaded wire sensor is as follows: the silicone wire sensor is threaded in one side of the nosepiece and out the other before the tubes are attached to the nosepiece. Potentially, a small hooked implement (not shown) can be inserted through oral prong 406, to snag sensor wire 404 and extract a portion of the wire out of the oral prong to form a loop. An insulator strip 408 is then inserted into the oral prong, between the two segments of wire, insulating the wire segments from one another and locking it in place, as well as sealing the oral prong to prevent pressure leaks. The side tubes are then inserted through the same holes on the sides of the nose piece and adhesive is applied to secure them in place, further securing the conductive wire.

Both of the abovementioned configurations share a number of similar features: in both cases the nosepiece is integrated with the temperature sensor, making the nosepiece a single integrated device/unit. The sensors are formed from temperature sensitive carbon-loaded polymeric material, that is to say that the material of both sensors is an electrically-conductive elastomer. The sensors are configured to have at least two sensitive regions, one for nasal flow and one for oral flow. In both configurations, the nosepiece body acts as an insulator which allows the use of non-insulated sensor elements (wire and ink) while maintaining galvanic insulation from the body of the user. The exposed loop of the wire sensors is tilted away from the mouth of the user, and therefore does not need to be insulated either. Both configurations utilize the elastic properties of the cannula nosepiece to facilitate a low cost connector used to connect the two ends of the sensor elements to the interface cable. In both configurations, the body of the nosepiece acts as a separator/insulator between the sensor element and the user, thereby facilitating the use of non-medically skin-contact approved sensor elements while still maintaining medical regulatory compliance (ISO 10993).

Yet another configuration is shown in FIG. 5. Innovative nosepiece 500 includes a nosepiece, two layers of electrically-conductive ink, and an insulation substrate separating the ink layers. A first conductive ink layer 504 is printed on the cannula surface of the nosepiece and oral prong. An insulation substrate 506 is laid over most of the area of the first conductive ink layer 504 (insulating the first conductive ink layer). A second conductive ink layer 502 (entirely visible in FIG. 5) is then printed over the insulation substrate 506. The insulation layer serves to insulate the first conductive ink layer 504 from the second conductive ink layer 502 forming a capacitor. Changes in capacitance between the two conductive ink layers, which occur due to changes in temperature, are measured and recorded.

In this configuration the second, external/distal layer 502 connects to a lead wire (not shown) on the left side of the nosepiece at connection area generally designated 508. The first, proximal layer 504, (of which only the externally exposed section is visible on the right side of the nosepiece) connects to a second lead wire (not shown), at the connection area generally designated 510, on the right side of the nosepiece. The two leads are routed along the cannula tubes over both ears and under the chin, feeding into an interface circuit, as above.

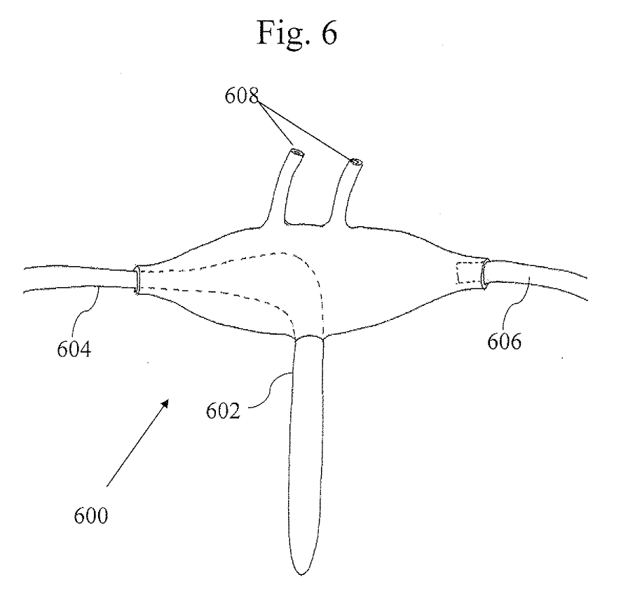

Yet another configuration is shown in FIG. 6. Innovative nosepiece 600 is operable to be connected with a first side tube 606 which is connected to a first transducer (not shown), as is commonly found in a standard nasal cannula arrangement. In this embodiment, the innovative temperature sensitive element is a closed volume section of a hollow tube 602, connected to a second pressure connecting tube 604 leading to a second pressure transducer (not shown). In this embodiment, temperature changes due to respiratory airflow over the closed tube section 602 generate pressure changes in the air volume locked inside the tube according the Boil's law of gasses. The pressure changes due to flow of air over the nose prongs 608 is routed through tube 606 on one end of the nosepiece, and the pressure changes due to temperature changes are routed through tube 604 on the other end of the nosepiece.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. An airflow sensor apparatus comprising:
(a) a nasal cannula nosepiece body;
(b) an oral prong extending from said nosepiece body; and
(c) a temperature sensor, integrated with said nasal cannula nosepiece body and said oral prong.

2. The apparatus of claim 1, wherein said temperature sensor includes an electrically-conductive elastomer based sensor material.

3. The apparatus of claim 2, wherein said electrically-conductive elastomer based sensor material is selected from the group of:
(i) an electrically-conductive ink;
(ii) an electrically-conductive Form-In-Place elastomer; and
(iii) an electrically-conductive elastomer wire.

4. The apparatus of claim 3, wherein said electrically-conductive ink sensor is printed on said nosepiece body and said oral prong.

5. The apparatus of claim 4, further comprising:
(d) a pair of electrically-conductive contact areas, conductively coupled to said printed thermal sensor, for conductively coupling mechanically attached external sensor leads to said printed thermal sensor.

6. The apparatus of claim 5, further comprising:
(d) a pair of circular abutments operationally coupled to a proximal surface of said nasal cannula nosepiece body, for intervening between said contact areas and a face of a user.

7. The apparatus of claim 4, further comprising:
(d) an insulation substrate, insulating said electrically-conductive ink thermal sensor; and
(e) a second electrically-conductive ink thermal sensor, printed on said insulation substrate, wherein said electrically-conductive ink thermal sensors and said insulation substrate combine to form a temperature-responsive capacitor.

8. The apparatus of claim 3, wherein said electrically-conductive ink material is mechanically compatible with said nosepiece body.

9. The apparatus of claim 1, wherein said temperature sensor includes a sealed volume tube threaded through said nosepiece body, and said oral prong, wherein said sealed volume tube is operable to be connected to a pressure transducer, and wherein temperature changes effect air pressure changes within said sealed volume tube, said air pressure changes operable to be measured by said pressure transducer.

10. A method of providing a device for simultaneous measurement of respiratory airflow pressure and temperature, comprising the steps of:
(a) providing a nasal cannula; and
(b) applying electrically-conductive, temperature sensitive, elastomer material to said nasal cannula.

11. The method of claim 10, wherein said electrically-conductive, temperature sensitive, elastomer material is selected from the group including:
(i) an electrically-conductive ink;
(ii) an electrically-conductive Form-In-Place elastomer; and
(iii) an electrically-conductive elastomer wire.

12. The method of claim 11, wherein said application is effected by a process selected from the group including:
(a) a pad printing process;
(b) a silk-screening process;
(c) direct placement of said electrically-conductive Form-In-Place elastomer material by a robotic 3D dispenser; and
(d) manual threading of said electrically-conductive elastomer wire through said nosepiece.
